(19) United States
(12) Patent Application Publication
(10) Pub. No.: US 2008/0009452 A1
(43) Pub. Date: Jan. 10, 2008

(54) PESTICIDAL COMPOSITION

(75) Inventors: Avner BARAZANI, Omer (IL); Jacob BARKAI, Moshav Nir (IL)

Correspondence Address: BROWDY AND NEIMARK, P.L.L.C.;624 NINTH STREET, NW, SUITE 300, WASHINGTON, DC, 20001-5303, US

(73) Assignee: MAKHTESHIM CHEMICAL WORKS LTD., Beer Sheva (IL)

(21) Appl. No.: 11/718,591

(22) PCT Filed: Oct. 31, 2005
(86) PCT No.: PCT/IL05/01139

§ 371 Date: May 3, 2007

(30) Foreign Application Priority Data
Nov. 4, 2004 (IL) 165021

(51) Int. Cl. A01N 47/28; A01N 43/40; A01N 43/04

(52) U.S. Cl. 514/28; 514/29; 514/340; 514/595

(57) ABSTRACT

The present invention provides an insecticidal composition comprising of at least one insecticidal compound with effective knock-down action selected from among group (A) comprising of imidacloprid, acetamiprid, thiamethoxam, thiacloprid, nitenpyram, dinotefuran, clothianidin, abamectin, emamectin, emamectin benzoate and fipronil, and at least one insecticidal compound with effective long-term action selected from among group (B) of insect growth regulators comprising of benzoylphenyl urea (BPU) compounds selected from among novaluron, lufenuron, hexaflumuron, triflumuron, diflubenzuron, chlorfluazuron, flufenoxuron, noviflumuron and teflubenzuron. Optionally, said insecticidal composition may further contain excipients and/or additives and/or surface active agents. Further provided is a method for insect control, comprising of applying at least one insecticidal compound with effective knock-down action selected from among group (A) comprising of imidacloprid, acetamiprid, thiamethoxam, thiacloprid, nitenpyram, dinotefuran, clothianidin, abamectin, emamectin, emamectin benzoate and fipronil, and at least one insecticidal compound with effective long-term action selected from among group (B) of insect growth regulators (IGR) comprising of benzoylphenyl urea (BPU) compounds; to a crop, agricultural product, plant matter, animal or locale.

### FIELD OF THE INVENTION

The present invention relates to the field of pesticidal compositions, particularly pesticidal composition containing at least two pesticidal active ingredients for improved control of pests.

### BACKGROUND OF THE INVENTION

In the practice of pest control, particularly insect control, there are two main factors which determine the effectiveness of the treatment; 1) immediate action on the pests (known in the art as “knock-down action”), 2) long term action (known also as “residual action”). Effective knock-down insecticides include pyrethroids, organic phosphoric acid esters, neonicotinoids, imidacloprid, acetamiprid and phenyl pyrazoles (fipronil). Effective long term insecticides include insect growth regulators (IGR) of various types, e.g. chitin synthesis inhibitors. Current methods for achieving the desired effect include repeated treatments over time with insecticides which have an effective knock-down action. The disadvantages of repeated treatments are; a) the use of relatively large doses of insecticides over time, which may create environmental problems, and b) there are certain periods within the life cycle of the crop when treatments with effective knock-down insecticides are prohibited since they may be absorbed in the crop or damage the crop. Using long term active insecticides may help in preventing repeated treatments, but may not be effective against certain insects which have reached a certain developmental stage in their life cycle. For example, the group of insecticides known as insect growth regulators (IGR) which generally have effective long term action are almost ineffective against adult insects due to the low knock-down effect.

Combination treatments of knock-down and long term insecticides have been reported. U.S. Pat. No. 6,685,954 reports on the effectiveness of fipronil in combination with IGR of the juvenile hormone mimic group in the treatment of ectoparasites in mammals. However, no publications were found which suggest such a combination for other purposes which are non-veterinary purposes. In other fields of applications such as in the filed of plants and plant matter, issues of phytotoxicity must be considered as well.

Accordingly, there is a long felt need for an insecticidal composition and method of pest control which will provide effective control of insects by providing effective knock-down and long term action, without creating environmental, toxic, phytotoxic and ecotoxic problems.

It is therefore a purpose of the present invention to provide an insecticidal composition which provides effective immediate and long term action against insects.

A further purpose of the present invention is to provide a method for pest control, particularly insect control, which provides effective immediate action and effective long term activity.

Other objects of the invention will become apparent as the description proceeds.

### SUMMARY OF THE INVENTION

The present invention provides an insecticidal composition comprising of at least one insecticidal compound with effective knock-down action selected from among group (A) comprising of imidacloprid, acetamiprid, thiamethoxam, thiacloprid, nitenpyram, dinotefuran, clothianidin, abamectin, emamectin, emamectin benzoate and fipronil, and at least one insecticidal compound with effective long-term action selected from among group (B) of insect growth regulators comprising of benzoylphenyl urea (BPU) compounds selected from among novaluron, lufenuron, hexaflumuron, triflumuron, diflubenzuron, chlorfluazuron, flufenoxuron, noviflumuron and teflubenzuron. Optionally, said insecticidal composition may further contain excipients and/or additives and/or surface active agents.

According to a further aspect of the present invention, there is provided a method for insect control, comprising of applying at least one insecticidal compound with effective knock-down action selected from among group (A) comprising of imidacloprid, acetamiprid, thiamethoxam, thiacloprid, nitenpyram, dinotefuran, clothianidin, abamectin, emamectin, emamectin benzoate and fipronil, and at least one insecticidal compound with effective long-term action selected from among group (B) of insect growth regulators (IGR) comprising of benzoylphenyl urea (BPU) compounds; to a crop, agricultural product, plant matter, animal or locale.

### DETAILED DESCRIPTION OF A PREFERRED EMBODIMENT OF THE INVENTION

The following description is illustrative of embodiments of the invention. The following description is not to be construed as limiting, it being understood that the skilled person may carry out many obvious variations to the invention.

Throughout the description, percentages of components are by weight, unless specifically noted differently. The terms “benzoyl urea”, “benzoylphenyl urea” and “BPU” as used throughout the specification are synonymous.

It has surprisingly been found that the combination of an insecticide with knock-down activity selected from among the groups of pesticides known in the art as neonicotinoids, nitromethylene-neonicotinoids and phenylpyrazoles denoted as group (A) comprising of imidacloprid, acetamiprid, thiamethoxam, thiacloprid, nitenpyram, dinotefuran, clothianidin, abamectin, emamectin, emamectin benzoate and fipronil, with an IGR insecticide selected from among the BPU group (B), provided a synergistic effect and effective insect control.

The long term IGR group of pesticides known as BPU herein designated “group (B)”, which includes inter alia, novaluron, lufenuron, hexaflumuron, triflumuron, diflubenzuron, chlorfluazuron, flufenoxuron, noviflumuron and teflubenzuron are suitable for the purposes of the present invention.

“Knock-down” pesticides suitable for the purposes of the present invention, herein designated “group (A)” include: acetamiprid, Imidacloprid, thiamethoxam, thiacloprid, nitenpyram, dinotefuran, clothianidin, abamectin, emamectin, emamectin benzoate and fipronil.

According to a preferred embodiment of the present invention there is provided an insecticidal composition comprising 0.1% to 60% of at least one knock down insecticide selected from among group (A); imidacloprid, acetamiprid, thiamethoxam, thiacloprid, nitenpyram, dinotefuran, clothianidin, abamectin, emamectin, emamectin benzoate and fipronil; and 0.5% to 60% of at least one long term insecticide selected from among an IGR insecticide group of BPU, group (B) comprising of novaluron, lufenuron, hexaflumuron, triflumuron, diflubenzuron, chlorfluazuron, flufenoxuron, noviflumuron and teflubenzuron. More preferably said composition contains 10% to 50% of at least one insecticide selected from group (A) and 5% to 15% of at least one insecticide selected from among group (B).

The composition of the present invention is formulated in a manner which suits the specific application Non-limiting examples of suitable formulations are; emulsion concentrates (EC), suspension concentrates (SC), water dispersible granules (WDG) and wettable powders (WP) and formulations suitable for oral, topical or injectable veterinary formulations.

According to yet a further embodiment of the compositions of the present invention, there is provided a synergistic insecticidal composition comprising of at least one knock-down insecticide selected from among group (A); imidacloprid, acetamiprid, thiamethoxam, thiacloprid, nitenpyram, dinotefuran, clothianidin, abamectin, emamectin, emamectin benzoate and fipronil, and of at least one long term insecticide selected from among an IGR insecticide of the group of BPU, group (B) comprising of novaluron, lufenuron, hexaflumuron, triflumuron, diflubenzuron, chlorfluazuron, flufenoxuron, noviflumuron and teflubenzuron, wherein the weight ratio between the group (A) insecticide and the group (B) insecticide is in the range of 1:100 to 100:1, more preferably 1:50 to 50:1.

The present invention further provides method for insect control, comprising of applying at least one insecticidal compound with effective knock-down action selected from among group (A) comprising of imidacloprid, acetamiprid, thiamethoxam, thiacloprid, nitenpyram, dinotefuran, clothianidin, abamectin, emamectin, emamectin benzoate and fipronil, and at least one insecticidal compound with effective long-term action selected from among group (B) of insect growth regulators (IGR) comprising of benzoylphenyl urea (BPU) compounds novaluron, lufenuron, hexaflumuron, triflumuron, diflubenzuron, chlorfluazuron, flufenoxuron, noviflumuron and teflubenzuron; to a crop, agricultural product, plant matter, animal or locale. The present method is effective for insect control in agricultural, horticultural, insect control in ornamentals, seed treatment and non-agricultural purposes, e.g. public health, storage and domestic use and veterinary.

According to a preferred embodiment of the method of the present invention, the application of the insecticides is simultaneous, separate or sequential application.

According to a preferred embodiment of the present method, the insecticidal composition of the present invention is employed for pest control.

According to specific embodiment of the method of the present invention when applied to plant matter, the insecticides of group (A) are applied at a rate of 1 g/hectar to 500 g/hectar and the insecticides of group (B) are applied at a rate of 1 g/hectar to 200 g/hectar. Preferably, group (A) insecticides are applied at rate of 100 g/hectar to 350 g/hectar and group (B) insecticides at a rate of 30 g/hectar to 100 g/hectar.

According to a specific embodiment of the method of the present invention, insecticides of group (A) are administered at a dosage of 0.1 to 50 mg/Kg body weight, and group (B) insecticides are administered at a dosage of 0.1 to 100 mg/Kg body weight. Preferably, the dosage for group (A) insecticides is 1 to 10 mg/Kg body weight and for group (B) insecticides 10 to 40 mg/Kg body weight. The administration according to the present method is topical, by injection or oral.

The present method and composition are particularly effective for controlling insects of, inter alia, the orders Lepidoptera, Coleoptera, Homoptera, Heteroptera, Diptera, Thysanoptera, Orthoptera, Anoplura, Siphonaptera, Mallophaga, Thysanura, Isoptera, Psocoptera and Hymenoptera, as well as representatives of the order Acarina of the families Ixodidae, Argasidae, Tetranychidae and Dermanyssidae.

In addition said method and composition are effective in controlling flies, e.g. Musca domestica, termites, cockroaches and mosquito larvae. The presently claimed method and composition is also suitable for controlling plant-destructive feeding insects in ornamentals and crops of useful plants, especially in cotton (e.g. against Spodoptera littoralis and Heliothis virescens) and in fruit and vegetables (e.g. against Laspeyresia pomonella, Cydia pomonella, Lithocolletis blancardella, Stigmella malella, Adoxophyes orana, Psylla piri, Cryptophlebia leucotreta, phyllocnistis citrella, Cydia molesta, Anarsia lineatella, Leptinotarsa decemlineata and Epilachna varivestis), as well as for controlling several species of mites, e.g., oleivora.

The present method and composition are further effective for controlling ectoparasites such as Lucilia sericata, in domestic animals and productive livestock, e.g. by treating animals, cowsheds, barns, stables, pastures and the like.

According to a particular embodiment of the present method, the pesticidal composition is used to prepare a tank mix which is then applied via spraying to the area, plant matter or crop needing treatment. The active ingredient concentration in the tank mix is adjusted to the particular application, depending on agricultural or non-agricultural application, the crop and the pest. Although various methods of application may be employed as the skilled artisan may appreciate, spraying is the preferred method of application. Various methods of application can also be employed when necessary. For example, in field crops systemic application via an irrigation system can be combined with foliar application.

According to a particular embodiment of the invention, separate application is employed wherein the knock down and long term insecticides are applied separately.

While embodiments of the invention have been described by way of illustration, it will be apparent that the invention may be carried out with many modifications, variations and adaptations, without departing from its spirit or exceeding the scope of the claims.

1. An insecticidal composition comprising of at least one insecticidal compound with effective knock-down action selected from among group (A) comprising of imidacloprid, acetamiprid, thiamethoxam, thiacloprid, nitenpyram, dinotefuran, clothianidin, abamectin, emamectin, emamectin benzoate and fipronil, and at least one insecticidal compound with effective long-term action selected from among group (B) of insect growth regulators comprising of benzoylphenyl urea (BPU) compounds, selected from among a group comprising of novaluron, lufenuron, hexaflumuron, triflumuron, diflubenzuron, chlorfluazuron, flufenoxuron, noviflumuron and teflubenzuron.

2. A composition according to claim 1 comprising 0.1% to 60% of at least one insecticide from group (A) and 0.5% to 60% of at least one insecticide from group (B), preferably 10% to 50% of at least one insecticide from group (A) and 5% to 15% of at least one insecticide from group (B).

3. A composition according to claim 1 wherein the weight to weight ratio between the group (A) and group (B) insecticides is in the range of 1:100 to 100:1, preferably 1:50 to 50:1.

4. A composition according to any of claims 1 to 3 wherein the group (A) insecticide is acetamiprid, imidacloprid or fipronil and the group (B) insecticide is novaluron.

5. A composition according to any of claims 1 to 4 wherein said insecticidal composition further contains excipients and/or additives and/or surface active agents.

6. A method for insect control, comprising of applying at least one insecticidal compound with effective knock-down action selected from among group (A) comprising of imidacloprid, acetamiprid, thiamethoxam, thiacloprid, nitenpyram, dinotefuran, clothianidin, abamectin, emamectin, emamectin benzoate and fipronil, and at least one insecticidal compound with effective long-term action selected from among group (B) of insect growth regulators (IGR) comprising of benzoylphenyl urea (BPU) compounds; to a crop, agricultural product, plant matter, animal or locale in need of insect control.

7. A method according to claim 6 wherein the application of the insecticides is simultaneous, separate or sequential.

8. A method according to claim 6 wherein when applied to plant matter, crop or field, the insecticides of group (A) are applied at a rate of 1 g/hectar to 500 g/hectar and the insecticides of group (B) are applied at a rate of 1 g/hectar to 200 g/hectar, preferably, group (A) insecticides are applied at rate of 100 g/hectar to 350 g/hectar and group (B) insecticides at a rate of 30 g/hectar to 100 g/hectar.

9. A method according to claim 6 wherein the application is to animals, the insecticides of group (A) are administered at a dosage of 0.1 to 50 mg/Kg body weight, and group (B) insecticides are administered at a dosage of 0.1 to 100 mg/Kg body weight, preferably, the dosage for group (A) insecticides is 1 to 10 mg/Kg body weight and for group (B) insecticides 10 to 40 mg/Kg body weight.

10. A method according to claim 9 wherein administration is topical, by injection or oral.
